(19) United States
(12) Patent Application Publication
(10) Pub. No.: US 2021/0093371 A1
(43) Pub. Date: Apr. 1, 2021

(54) MEDICAL DEVICES AND METHOD

(71) Applicant: RELIGN Corporation, Campbell, CA (US)

(72) Inventors: Aaron Germain, San Jose, CA (US); Jeff Norton, Emerald Hills, CA (US)

(73) Assignee: RELIGN Corporation, Campbell, CA (US)

(21) Appl. No.: 17/020,539

(22) Filed: Sep. 14, 2020

Related U.S. Application Data
(60) Provisional application No. 62/908,425, filed on Sep. 30, 2019.

(51) Int. Cl. A61B 18/14

(52) U.S. Cl. CPC A61B 18/14; A61B 2018/00601

(57) ABSTRACT

A tissue treatment device includes a sleeve assembly having an outer sleeve and an inner sleeve co-axially and rotatably received in an axial lumen of the outer sleeve. A tapered ceramic member has a cutting window formed on a side surface thereof and is attached to a distal end of the outer member. A distal electrode has at least one serrated electrode surface disposed along at least one axially aligned edge and is disposed in the cutting window of the tapered ceramic member so that said at least one serrated electrode surface passes across the cutting window as the inner sleeve rotated in the outer sleeve. A hub is attached to a proximal end of the sleeve assembly and is configured to be detachably received in a motorized handle.

### CROSS-REFERENCE TO RELATED APPLICATIONS

This application claims the benefit of U.S. Provisional No. 62/908,425 (Attorney Docket No. 41879-749.101), filed Sep. 30, 2019, the entire content of which is incorporated herein by reference.

### BACKGROUND OF THE INVENTION

### 1. Field of the Invention

This invention relates to tissue cutting and removal systems wherein a motor-driven electrosurgical device is provided for cutting and removing tissue from a patient's body.

In several surgical procedures including arthroscopy, spine procedures and ENT, there is a need for cutting and removal of hard and soft tissues. In particular, it would be desirable to provide cutters capable of removing and extracting tissue from spinal disks and other orthopedic and non-orthopedic applications.

### 2. Description of the Background Art

Commonly owned U.S. Pat. No. 10,582,966 describe an elongated shaft assembly that includes a rotatable inner cutting sleeve and a non-rotating outer sleeve having a window of the inner cutting sleeve which is selectively rotatable within an opening of the non-rotating outer sleeve.

### SUMMARY OF THE INVENTION

In a first aspect, the present invention provides a tissue treatment device comprising a sleeve assembly having an outer sleeve and an inner sleeve co-axially and rotatably received in an axial lumen of the outer sleeve. A tapered ceramic member has a cutting window formed on a side surface thereof attached is attached to a distal end of the outer member. A distal electrode has at least one serrated electrode surface disposed along at least one axially aligned edge thereof so that said at least one serrated electrode surface passes across the cutting window in the tapered ceramic member as the inner sleeve rotated in the outer sleeve. A hub is attached to a proximal end of the sleeve assembly and is configured to be detachably received in a motorized handle.

In specific instances, the tapered ceramic member may have a generally conical shape, and the cutting window may have an ovoid periphery. In further specific instances, the distal electrode may have an axial backbone with the at least one serrated electrode surface which may be disposed along at least one axially side thereof, optionally have one such electrode surface on each of the two axially sides of the electrode, and further optionally having a symmetric structure. The axial backbone may be curved, optionally in order to conform to an inner curved surface of the tapered ceramic member as the electrode is rotated.

In specific instances, the at least one serrated electrode surface of the tissue treatment device may have an active area no greater than 10 mm² and/or no less than 1 mm². Typically, the active area may be in any one of the following ranges: 1 mm² to 10 mm²; 1 mm² to 8 mm²; 1 mm² to 6 mm²; 2 mm² to 10 mm²; 2 mm² to 8 mm²; and 2 mm² to 6 mm².

The tissue treatment devices of the present invention as just described may be incorporated into surgical systems further comprising a handle including a motor attachable to the hub, where the motor is typically configured to rotatably drive the inner sleeve relative to the outer sleeve, a radiofrequency (RF) current source configured to be coupled to the at least one distal electrode, and a controller configured to be operatively coupled to the motor in the handle and to the RF source.

In a second aspect, the present invention provides methods for performing a discectomy in a patient. Such methods typically comprise providing a tissue treatment device as generally described above, typically including a sleeve assembly having an outer sleeve and an inner sleeve co-axially and rotatably received in an axial lumen of the outer sleeve, a tapered ceramic member having a cutting window formed on a side surface thereof attached to a distal end of the outer member, and a distal electrode having at least one serrated electrode surface disposed along at least one axially aligned edge thereof so that said at least one serrated electrode surface passes across the cutting window in the tapered ceramic member as the inner sleeve rotated in the outer sleeve. The methods further comprise advancing the tapered ceramic member into a spinal disc of the patient. The inner sleeve is rotated relative to the outer sleeve to advance the at least one serrated electrode surface past the cutting window, and radiofrequency current is applied to the at least one serrated electrode surface to ablate or otherwise cut or resect tissue of the disc as the inner sleeve is being rotated.

In some instances, the inner sleeve may be rotated in one direction only. In other instances, the inner sleeve may be rotated in two direction. In still other instances, the inner sleeve may be rotationally oscillated.

In exemplary aspects of these methods, the tapered ceramic member may have a generally conical shape and the cutting window may have an ovoid periphery. The distal electrode may have an axial backbone with the at least one serrated electrode surface disposed along at least one axially side thereof, and the axial backbone may be curved to conform to an inner curved surface of the tapered ceramic member as the electrode is rotated. The distal electrode may include two serrated electrode surfaces disposed symmetrically on each lateral side of the axial backbone. The at least one serrated electrode surface may have an active area no greater than 10 mm² and/or no less than 1 mm². Often, the active area will be one of the following ranges: 1 mm² to 10 mm²; 1 mm² to 8 mm²; 1 mm² to 6 mm²; 2 mm² to 10 mm²; 2 mm² to 8 mm²; and 2 mm² to 6 mm².

### BRIEF DESCRIPTION OF THE DRAWINGS

Various embodiments of the present invention will now be discussed with reference to the appended drawings. It should be appreciated that the drawings depict only typical embodiments of the invention and are therefore not to be considered limiting in scope.

FIG. 1 is a perspective view of an arthroscopic cutting system that includes reusable handle with a motor drive and a detachable single-use cutting probe, wherein the cutting probe is shown in two orientations as it may be coupled to the handle with the probe and working end in upward orientation or a downward orientation relative to the handle, and wherein the handle includes an LCD screen for displaying operating parameters of system during use together with control actuators on the handle.

FIG. 2A is an enlarged longitudinal sectional view of the hub of the probe of FIG. 1 taken along line 2A-2A of FIG. 1 with the hub and probe in an upward orientation relative to the handle, further showing Hall effect sensors carried by the handle and a plurality of magnets carried by the probe hub for device identification, for probe orientation and determining the position of motor driven components of the probe relative to the handle.

FIG. 2B is a sectional view of the hub of FIG. 1 taken along line 2B-2B of FIG. 1 with the hub and probe in a downward orientation relative to the handle showing the Hall effect sensor and magnets having a different orientation compared to that of FIG. 2A.

FIG. 3A is an enlarged perspective view of the working end of the probe of FIG. 1 in an upward orientation with the rotatable cutting member in a first position relative to the outer sleeve wherein the window in the cutting member is aligned with the window of the outer sleeve.

FIG. 3B is a perspective view of the working end of FIG. 1 in an upward orientation with the rotatable cutting member in a second position relative to the outer sleeve wherein the electrode carried by the cutting member is aligned with a centerline of the window of the outer sleeve.

FIG. 4 is a perspective view of a working end of a variation of a probe that may be detachably coupled to the handle of FIG. 1, wherein the working end includes a bone burr extending distally from the outer sleeve.

FIG. 5 is a perspective view of a working end of a variation of a probe that may be detachably coupled to the handle of FIG. 1, wherein the working end has a reciprocating electrode.

FIG. 6 is a perspective view of a working end of another variation of a probe that may be detachably coupled to the handle of FIG. 1, wherein the working end has a hook electrode that has extended and non-extended positions.

FIG. 7 is a perspective view of a working end of yet another variation of a probe that may be detachably coupled to the handle of FIG. 1, wherein the working end has an openable-closeable jaw structure for cutting tissue.

FIG. 8 is a chart relating to set speeds for a probe with a rotating cutting member as in FIGS. 1 and 3A that schematically shows the method used by a controller algorithm for stopping rotation of the cutting member in a selected default position.

FIG. 9A is a longitudinal sectional view of a probe hub that is similar to that of FIG. 2A, except the hub of FIG. 9A has an internal cam mechanism for converting rotational motion to linear motion to axially reciprocate an electrode as in the working end of FIG. 5, wherein FIG. 9A illustrated the magnets in the hub and drive coupling are the same as in FIG. 2A and the hub is in an upward facing position relative to the handle.

FIG. 9B is a sectional view of the hub of FIG. 9A rotated 180° in a downward facing position relative to the handle.

FIG. 10 is a perspective view of a working end of yet another variation of a probe that may be detachably coupled to the handle of FIG. 1, wherein the working end has an outer ceramic member and a rotatable inner cutting electrode that is adapted for rotation within a window in the ceramic outer member.

FIG. 11A is a perspective view of the rotatable cutting electrode of FIG. 10 separated from ceramic outer member.

FIG. 11A-1 is an enlarged view of the rotatable cutting electrode of FIG. 11A.

FIG. 11B is a perspective view of the ceramic outer member of FIG. 10 separated from the rotating inner electrode member.

FIG. 12 is a schematic sectional view of a patient's nasal cavities showing the working end of FIG. 10 being used to remove turbinate tissue.

FIG. 13 is a schematic partial sectional view of a patient's spinal disc showing the working end of a device similar to that of FIG. 10 being used to remove the nucleus of the disc.

FIG. 14 is a schematic view of a patient's spine showing the working end of device similar to that of FIG. 7 being used in a microdiscectomy procedure.

FIG. 15 is a side view of a working end of yet another variation of a probe that may be detachably coupled to the handle of FIG. 1, wherein the working end has an electrode arrangement for ablating tissue and wherein the working end can be articulated with the motor drive.

FIG. 16 is a schematic view of a patient's spine showing the working end of device of FIG. 15 being used to treat or ablate a spinal tumor.

### DETAILED DESCRIPTION OF THE INVENTION

The present invention relates to bone cutting and tissue removal devices and related methods of use. Several variations of the invention will now be described to provide an overall understanding of the principles of the form, function and methods of use of the devices disclosed herein. In general, the present disclosure provides for variations of arthroscopic tools adapted for cutting bone, soft tissue, meniscal tissue, and for RF ablation and coagulation. The arthroscopic tools are typically disposable and are configured for detachable coupling to a non-disposable handle that carries a motor drive component. This description of the general principles of this invention is not meant to limit the inventive concepts in the appended claims.

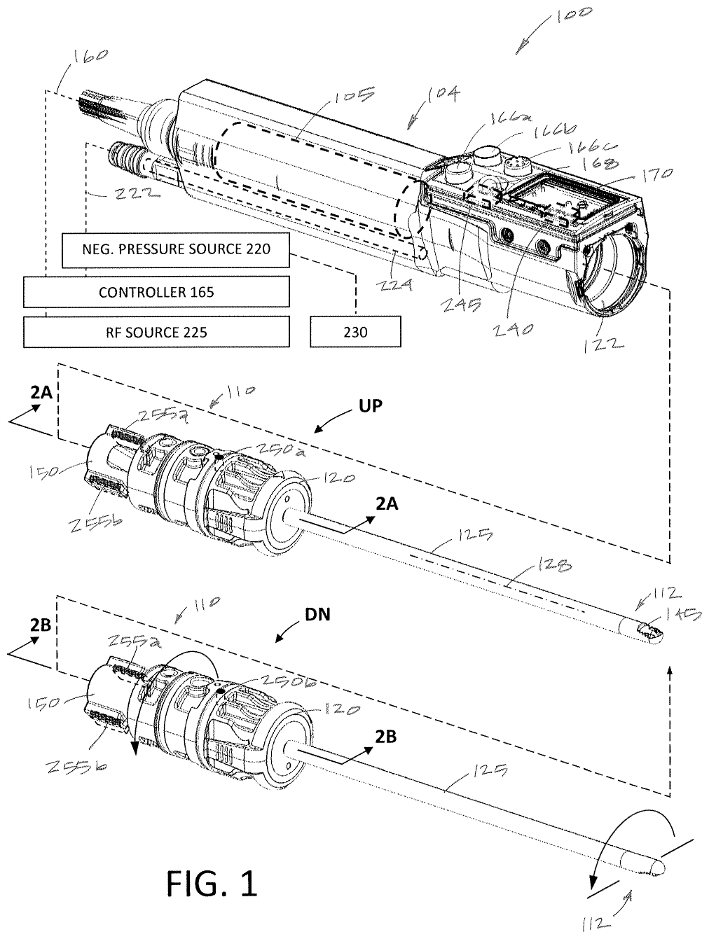

In one variation shown in FIG. 1, the arthroscopic system 100 of the present invention provides a handle 104 with motor drive 105 and a disposable shaver assembly or probe 110 with a proximal hub 120 that can be received by receiver or bore 122 in the handle 104. In one aspect, the probe 110 has a working end 112 that carries a high-speed rotating cutter that is configured for use in many arthroscopic surgical applications, including but not limited to treating bone in shoulders, knees, hips, wrists, ankles and the spine as well as for ENT procedures.

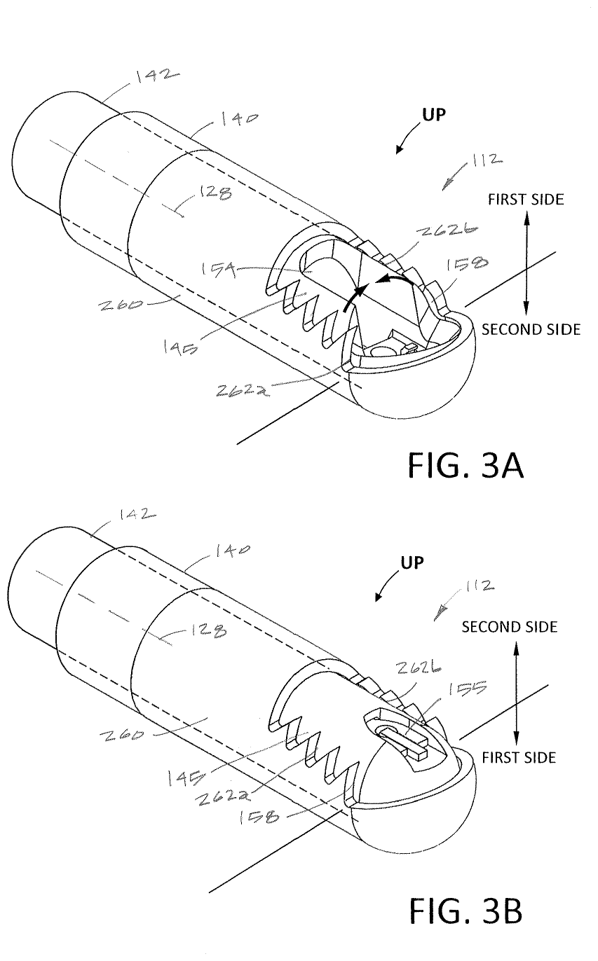

In FIGS. 1, 2A and 3A, it can be seen that probe 110 has a shaft 125 extending along longitudinal axis 128 that comprises an outer sleeve 140 and an inner sleeve 142 rotatably disposed therein with the inner sleeve 142 carrying a distal ceramic cutting member 145 (FIG. 3A). The shaft 125 extends from the proximal hub 120 wherein the outer sleeve 140 is coupled in a fixed manner to the hub 120 which can be an injection molded plastic, for example, with the outer sleeve 140 insert molded therein. The inner sleeve 142 is coupled drive coupling 150 that is configured for coupling to the rotating motor shaft 152 of motor drive unit 105. More in particular, the rotatable cutting member 145 that is fabricated of a ceramic material with sharp cutting edges on opposing sides 152a and 152b of window 154 therein for cutting soft tissue. The motor drive 105 is operatively coupled to the ceramic cutter to rotate the cutting member at speeds ranging from 1,000 rpm to 20,000 rpm. In FIG. 3B, it can be seen that cutting member 145 also carries an RF electrode 155 in a surface opposing the window 154. The cutting member 145 rotates and shears tissue in the toothed opening or window 158 in the outer sleeve 140 (FIG. 3A). A probe of the type shown in FIG. 1 is described in more detail in co-pending and commonly owned patent application Ser. No. 15/421,264 filed Jan. 31, 2017 (Atty. Docket 41879-714.201) titled ARTHROSCOPIC DEVICES AND METHODS which is incorporated herein in its entirety by this reference.

As can be seen in FIG. 1, the probe 110 is shown in two orientations for detachable coupling to the handle 104. More particularly, the hub 120 can be coupled to the handle 104 in an upward orientation indicated at UP and a downward orientation indicated at DN where the orientations are 180° opposed from one another. It can be understood that the upward and downward orientations are necessary to orient the working end 112 either upward or downward relative to the handle 104 to allow the physician to interface the cutting member 145 with targeted tissue in all directions without having to manipulate the handle in 360° to access tissue.

In FIG. 1, it can be seen that the handle 104 is operatively coupled by electrical cable 160 to a controller 165 which controls the motor drive unit 105. Actuator buttons 166a, 166b or 166c on the handle 104 can be used to select operating modes, such as various rotational modes for the ceramic cutting member 145. In one variation, a joystick 168 be moved forward and backward to adjust the rotational speed of the ceramic cutting member 145. The rotational speed of the cutter can continuously adjustable or can be adjusted in increments up to 20,000 rpm. An LCD screen 170 is provided in the handle for displaying operating parameters, such as cutting member RPM, mode of operation, etc.

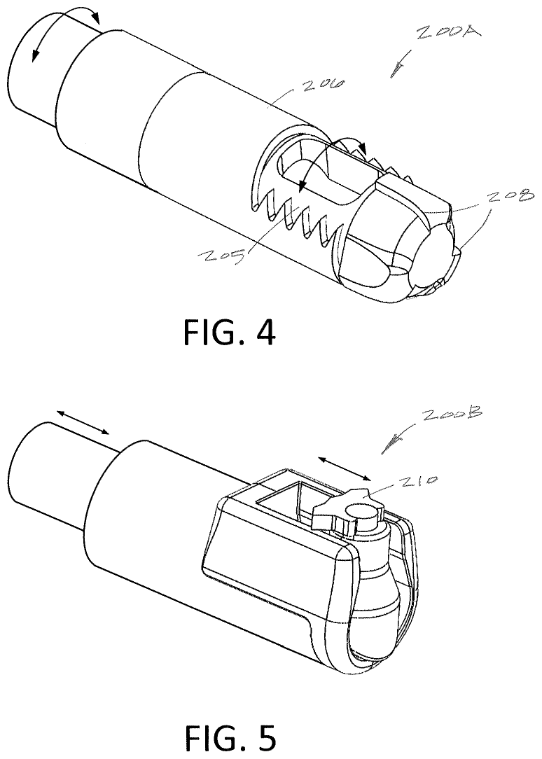

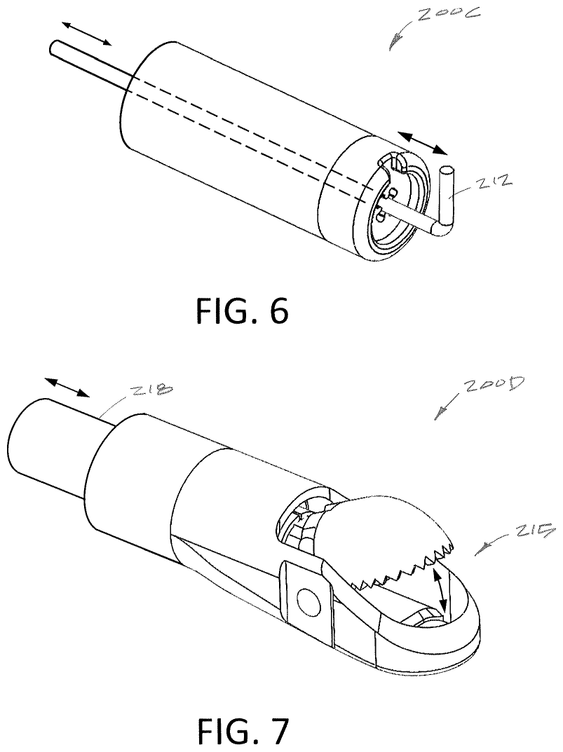

It can be understood from FIG. 1 that the system 100 and handle 104 is adapted for use with various disposable probes which can be designed for various different functions and procedures For example, FIG. 4 illustrates a different variation of a probe working end 200A that is similar to working end 112 of probe 110 of FIGS. 3A-3B, except the ceramic cutting member 205 extends distally from the outer sleeve 206 and the cutting member has burr edges 208 for cutting bone. The probe of FIG. 4 is described in more detail in co-pending and commonly owned patent application Ser. No. 15/271,184 filed Sep. 20, 2016 (Atty. Docket 41879-728.201) titled ARTHROSCOPIC DEVICES AND METHODS. FIG. 5 illustrates a different variation of a probe working end 200B with a reciprocating electrode 210 in a type of probe described in more detail in co-pending and commonly owned patent application Ser. No. 15/410,723 filed Jan. 19, 2017 (Atty. Docket 41879-713.201) titled ARTHROSCOPIC DEVICES AND METHODS. In another example, FIG. 6 illustrates another variation of a probe working end 200C that has an extendable-retractable hook electrode 212 in a probe type described in more detail in co-pending and commonly owned patent application Ser. No. 15/454,342 filed Mar. 9, 2017 (Atty. Docket 41879-715.201) titled ARTHROSCOPIC DEVICES AND METHODS. In yet another example, FIG. 7 illustrates a variation of a working end 200D in a probe type having an openable-closable jaw structure 215 actuated by reciprocating member 218 for trimming meniscal tissue, spine tissue or other tissue as described in more detail in co-pending and commonly owned patent application Ser. No. 15/483,940 filed Apr. 10, 2017 (Atty. Docket 41879-721.201) titled ARTHROSCOPIC DEVICES AND METHODS. All of the probes of FIGS. 4-7 can have a hub similar to hub 120 of probe 110 of FIG. 1 for coupling to the same handle 104 of FIG. 1, with some of the probes (see FIGS. 5-7) having a hub mechanism for converting rotational motion to linear motion. All of the patent applications just identified in this paragraph are incorporated herein by this reference.

FIG. 1 further shows that the system 100 also includes a negative pressure source 220 coupled to aspiration tubing 222 which communicates with a flow channel 224 in handle 104 and can cooperate with any of the probes 110, 200A, 200B or 200C of FIGS. 1-3B, 4, 5 and 6. In FIG. 1 it also can be seen that the system 100 includes an RF source 225 which can be connected to an electrode arrangement in any of the probes 110, 200A, 200B or 200C of FIGS. 1-3B, 4, 5 and 6. The controller 165 and microprocessor therein together with control algorithms are provided to operate and control all functionality, which includes controlling the motor drive 105 to move a motor-driven component of any probe working end 110, 200A, 200B or 200C, as well as for controlling the RF source 225 and the negative pressure source 220 which can aspirate fluid and tissue debris to collection reservoir 230.

As can be understood from the above description of the system 100 and handle 104, the controller 165 and controller algorithms need to be configured to perform and automate many tasks to provide for system functionality. In a first aspect, controller algorithms are needed for device identification so that when any of the different probes types 110, 200A, 200B, 200C or 200D of FIGS. 1 and 4-7 are coupled to handle 104, the controller 165 will recognize the probe type and then select algorithms for operating the motor drive 105, RF source 225 and negative pressure source 220 as is needed for the particular probe. In a second aspect, the controller is configured with algorithms that identify whether the probe is coupled to the handle 104 in an upward or downward orientation relative to the handle, wherein each orientation requires a different subset of the operating algorithms. In another aspect, the controller has separate control algorithms for each probe type wherein some probes have a rotatable cutter while others have a reciprocating electrode or jaw structure. In another aspect, most if not all the probes 110, 200A, 200B, 200C and 200D (FIGS. 1, 4-7) require a default “stop” position in which the motor-driven component is stopped in a particular orientation within the working end. For example, a rotatable cutter 145 with an electrode 155 needs to have the electrode centered within an outer sleeve window 158 in a default position such as depicted in FIG. 3B. Some of these systems, algorithms and methods of use are described next.

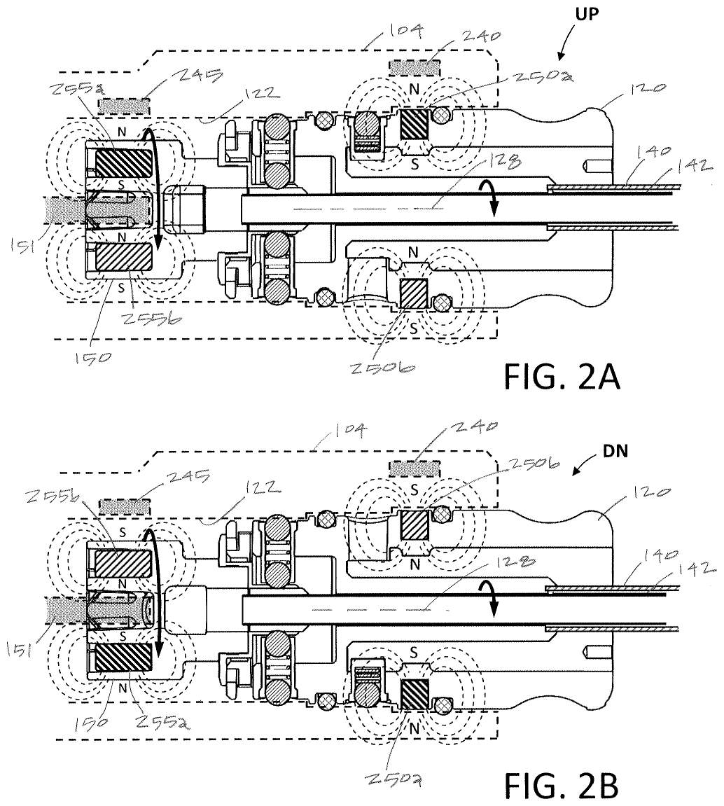

Referring to FIGS. 1 and 2A-2B, it can be seen that handle 104 carries a first Hall effect sensor 240 in a distal region of the handle 104 adjacent the receiving passageway 122 that receives the hub 120 of probe 110. FIG. 2A corresponds to the probe 110 and working end 112 in FIG. 1 being in the upward orientation indicated at UP. FIG. 2B corresponds to probe 110 and working end 112 in FIG. 1 being in the downward orientation indicated at DN. The handle 104 carries a second Hall effect sensor 245 adjacent the rotatable drive coupling 150 of the probe 110. The probe 110 carries a plurality of magnets as will be described below that interact with the Hall effect sensors 240, 245 to provide multiple control functions in cooperation with controller algorithms, including (i) identification of the type of probe coupled to the handle, (ii) the upward or downward orientation of the probe hub 120 relative to the handle 104, and (iii) the rotational position and speed of rotating drive collar 150 from which a position of either rotating or reciprocating motor-driven components can be determined.

The sectional views of FIGS. 2A-2B show that hub 120 of probe 110 carries first and second magnets 250a and 250b in a surface portion thereof. The Hall sensor 240 in handle 104 is in axial alignment with either magnet 250a or 250b when the probe hub 120 is coupled to handle 104 in an upward orientation (FIGS. 1 and 2A) or a downward orientation (FIGS. 1 and 2B). In one aspect as outlined above, the combination of the magnets 250a and 250b and the Hall sensor 240 can be used to identify the probe type. For example, a product portfolio may have from 2 to 10 or more types of probes, such as depicted in FIGS. 1 and 4-7, and each such probe type can carry magnets 250a, 250b having a specific, different magnetic field strength. Then, the Hall sensor 240 and controller algorithms can be adapted to read the magnetic field strength of the particular magnet(s) in the probe which can be compared to a library of field strengths that correspond to particular probe types. Then, a Hall identification signal can be provided to the controller 165 to select the controller algorithms for operating the identified probe, which can include parameters for operating the motor drive 105, negative pressure source 220 and/or RF source 225 as may be required for the probe type. As can be seen in FIGS. 1, 2A and 2B, the probe hub 120 can be coupled to handle 104 in upward and downward orientations, in which the North (N) and South (S) poles of the magnets 250a, 250b are reversed relative to the probe axis 128. Therefore, the Hall sensor 240 and associated algorithms look for magnetic field strength regardless of polarity to identify the probe type.

Referring now to FIGS. 1, 2A-2B and 3A-3B, the first and second magnets 250a and 250b with their different orientations of North (N) and South (S) poles relative to central longitudinal axis 128 of hub 120 are also used to identify the upward orientation UP or the downward orientation DN of hub 120 and working end 112. In use, as described above, the physician may couple the probe 110 to the handle receiving passageway 122 with the working end 112 facing upward or downward based on his or her preference and the targeted tissue. In can be understood that controller algorithms adapted to stop rotation of the cutting member 145 in the window 158 of the outer sleeve 104 of working end 112 need to “learn” whether the working end is facing upward or downward, because the orientation or the rotating cutting member 145 relative to the handle and Hall sensor 240 would vary by 180°. The Hall sensor 240 together with a controller algorithm can determine the orientation UP or the downward orientation DN by sensing whether the North (N) or South (S) pole of either magnet 250a or 250b is facing upwardly and is proximate the Hall sensor 240.

In another aspect of the invention, in probe 110 (FIG. 1) and other probes, the motor-driven component of a working end, such as rotating cutter 145 of working end 112 of FIGS. 1 and 3A-3B needs to stopped in a selected rotational position relative to a cut-out opening or window 158 in the outer sleeve 140. Other probe types may have a reciprocating member or a jaw structure as described above, which also needs a controller algorithm to stop movement of a moving component in a selected position, such as the axial-moving electrodes of FIGS. 5-6 and the jaw structure of FIG. 7. In all probes, the motor drive 105 couples to the rotating drive coupling 150, thus sensing the rotational position of the drive coupling 150 can be used to determine the orientation of the motor-driven component in the working end. More in particular, referring to FIGS. 1 and 2A-2B, the drive coupling 150 carries third and fourth magnets 255a or 255b with the North (N) and South (S) poles of magnets 255a or 255b being reversed relative to the probe axis 128. Thus, Hall sensor 245 can sense when each magnet rotates passes the Hall sensor and thereby determine the exact rotational position of the drive coupling 150 twice on each rotation thereof (once for each magnet 255a, 255b). Thereafter, a controller tachometer algorithm using a clock can determine and optionally display the RPM of the drive coupling 150 and, for example, the cutting member 145 of FIG. 3A.

In another aspect of the invention, the Hall sensor 245 and magnets 255a and 255b (FIGS. 1 and 2A) are used in a set of controller algorithms to stop the rotation of a motor-driven component of a working end, for example, cutting member 145 of FIGS. 1 and 3A-3B in a pre-selected rotational position. In FIG. 3A, it can be seen that the inner sleeve 142 and a “first side” of cutting member 145 and window 154 therein is stopped and positioned in the center of window 158 of outer sleeve 140. The stationary position of cutting member 145 and window 154 in FIG. 3A may be used for irrigation or flushing of a working space to allow for maximum fluid outflow through the probe.

FIG. 3B depicts inner sleeve 142 and a “second side” of cutting member 145 positioned about the centerline of window 158 in the outer sleeve 140. The stationary or stopped position of cutting member 145 in FIG. 3B is needed for using the RF electrode 155 to ablate or coagulate tissue. It is important that the electrode 155 is maintained along the centerline of the outer sleeve window 158 since the outer sleeve 140 typically comprises return electrode 260. The position of electrode 155 in FIG. 3B is termed herein a “centerline default position”. If the cutting member 145 and electrode 155 were rotated so as to be close to an edge 262a or 262b of window 158 in outer sleeve 140, RF current could arc between the electrodes 155 and 260 and potentially cause a short circuit disabling the probe. Therefore, a robust and reliable stop mechanism is required which is described next.

As can be understood from FIGS. 1 and 2A-2B, the controller 165 can always determine in real time the rotational position of drive coupling 150 and therefore the angular or rotational position of the ceramic cutting member 145 and electrode 155 can be determined. A controller algorithm can further calculate the rotational angle of the electrode 155 away from the centerline default position as the Hall sensor 245 can sense lessening of magnetic field strength as a magnet 255a or 255b in the drive coupling 150 rotates the electrode 155 away from the centerline default position. Each magnet has a specified, known strength and the algorithm can use a look-up table with that lists fields strengths corresponding to degrees of rotation away from the default position. Thus, if the Hall signal responsive to the rotated position of magnet 255a or 255b drops a specified amount from a known peak value in the centerline default position, it means the electrode 155 has moved away from the center of the window 158. In one variation, if the electrode 155 moves a selected rotational angle away from the centerline position during RF energy delivery to the electrode, the algorithm turns off RF current instantly and alerts the physician by an aural and/or visual signal, such as an alert on the LCD screen 170 on handle 104 and/or on a screen on a controller console (not shown). The termination of RF current delivery thus prevents the potential of an electrical arc between electrode 155 and the outer sleeve electrode 260.

It can be understood that during use, when the electrode 155 is in the position shown in FIG. 3B, the physician may be moving the energized electrode over tissue to ablate or coagulate tissue. During such use, the cutting member 145 and electrode 155 can engage or catch on tissue which inadvertently rotate the electrode 155 out of the default centerline position. Therefore, the system provides a controller algorithm, herein called an “active electrode monitoring” algorithm, wherein the controller continuously monitors Hall position signals from sensor 245 during RF energy delivery in both an ablation mode and a coagulation mode to determine if the electrode 155 and inner sleeve 142 have been bumped off the centerline position. In a variation, the controller algorithms can be configured to then re-activate the motor drive 105 to move the inner sleeve 142 and electrode 155 back to the default centerline position sleeve if electrode 155 had been bumped off the centerline position. In another variation, the controller algorithms can be configured to again automatically deliver RF current to RF electrode 155 when it is moved back to the to the default centerline position. Alternatively, the controller 165 can require the physician to manually re-start the delivery of RF current to the RF electrode 155 when it is moved back to the to the centerline position. In an aspect of the invention, the drive coupling 150 and thus magnets 255a and 255b are attached to inner sleeve 142 and cutting member 145 in a pre-determined angular relationship relative to longitudinal axis 128 so that the Hall signal responsive to magnets 255a, 255b is the same for all probes within a probe type to thus allow the controller algorithm to function properly.

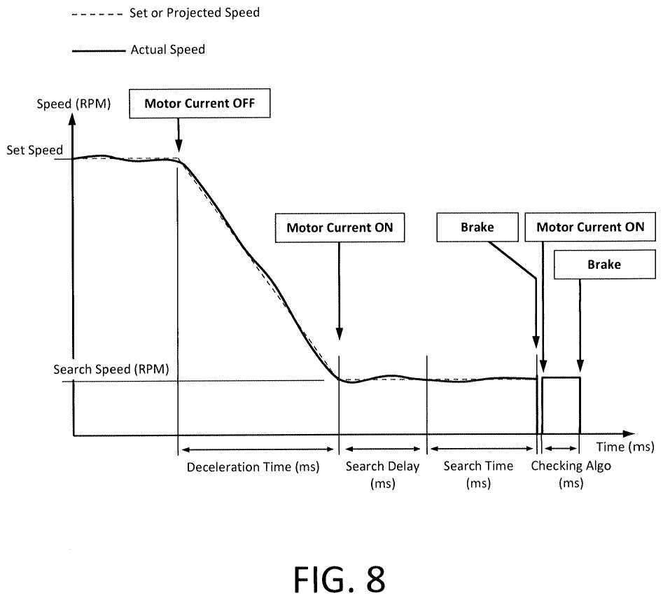

Now turning to the stop mechanism or algorithms for stopping movement of a motor-driven component of working end 112, FIG. 8 schematically illustrates the algorithm and steps of the stop mechanism. In one variation, referring to FIG. 8, the stop mechanism corresponding to the invention uses (i) a dynamic braking method and algorithm to stop the rotation of the inner sleeve 142 and cutting member 145 (FIGS. 1, 3A-3B) in an initial position, and thereafter (ii) a secondary checking algorithm is used to check the initial stop position that was attained with the dynamic braking algorithm, and if necessary, the stop algorithm can re-activate the motor drive 105 to slightly reverse (or move forward) the rotation of drive coupling 150 and inner sleeve 142 as needed to position the cutting member 145 and electrode 155 within at the centerline position or within 0° to 5° of the targeted centerline default position. Dynamic braking is described further below. FIG. 8 schematically illustrates various aspects of controller algorithms for controlling the rotational speed of the cutting member and for stopping the cutting member 145 in the default centerline position.

In FIG. 8, it can be understood that the controller 165 is operating the probe 110 of FIGS. 1 and 3A-3B at a “set speed” which may be a PID controlled, continuous rotation mode in one direction or may be an oscillating mode where the motor drive 105 rotates the cutting member 145 in one direction and then reverses rotation as is known in the art. At higher rotational speeds such as 1,000 RPM to 20,000 RPM, it is not practical or feasible to acquire a signal from Hall sensor 245 that indicates the position of a magnet 255a or 255b in the drive coupling 150 to apply a stop algorithm. In FIG. 8, when the physician stop cutting with probe 110 by releasing actuation of an actuator button or foot pedal, current to the motor drive 105 is turned off. Thereafter, the controller algorithm uses the Hall sensor 245 to monitor deceleration of rotation of the drive coupling 150 and inner sleeve 142 until a slower RPM is reached. The deceleration period may be from 10 ms to 1 sec and typically is about 100 ms. When a suitable slower RPM is reached which is called a “search speed” herein (see FIG. 8), the controller 165 re-activates the motor drive 105 to rotate the drive coupling at a low speed ranging from 10 RPM to 1,000 RPM and in one variation is between 50 RPM and 250 RPM. An initial “search delay” period ranging from 50 ms to 500 ms is provided to allow the PID controller to stabilize the RPM at the selected search speed. Thereafter, the controller algorithm monitors the Hall position signal of magnet strength and when the magnet parameter reaches a predetermined threshold, for example, when the rotational position of drive coupling 150 and electrode 155 correspond to the centerline default position of FIG. 3B, the control algorithm then applies dynamic braking to instantly stop rotation of the motor drive shaft 152, drive coupling 150 and the motor-driven component of the probe. FIG. 8 further illustrates that the controller can check the magnet/drive coupling 150 position after the braking and stopping steps. If the Hall position signal indicates that the motor-driven component is out of the targeted default position, the motor drive 105 can be re-activated to move the motor-driven component and thereafter the brake can be applied again as described above.

Dynamic braking as shown schematically in FIG. 8 may typically stop the rotation of the drive coupling 150 with a variance of up to about 0°-15° of the targeted stop position, but this can vary even further when different types of tissue are being cut and impeding rotation of the cutting member 145, and also depending on whether the physician has completely disengaged the cutting member from the tissue interface when the motor drive is de-activated. Therefore, dynamic braking alone may not assure that the default or stop position is within a desired variance.

As background, the concept of dynamic braking is described in the following literature: https://www.ab.com/support/abdrives/documentation/techpapers/RegenOverview01.pdf and http://literature.rockwellautomation.com/idc/groups/literature/documents/wp/drives-wp004_-en-p.pdf. Basically, a dynamic braking system provides a chopper transistor on the DC bus of the AC PWM drive that feeds a power resistor that transforms the regenerative electrical energy into heat energy. The heat energy is dissipated into the local environment. This process is generally called dynamic braking with the chopper transistor and related control and components called the chopper module and the power resistor called the dynamic brake resistor. The entire assembly of chopper module with dynamic brake resistor is sometimes referred to as the dynamic brake module. The dynamic brake resistor allows any magnetic energy stored in the parasitic inductance of that circuit to be safely dissipated during the turn off of the chopper transistor.

The method is called dynamic braking because the amount of braking torque that can be applied is dynamically changing as the load decelerates. In other words, the braking energy is a function of the kinetic energy in the spinning mass and as it declines, so does the braking capacity. So the faster it is spinning or the more inertia it has, the harder you can apply the brakes to it, but as it slows, you run into the law of diminishing returns and at some point, there is no longer any braking power left.

In another aspect of the invention, a method has been developed to increase the accuracy of the stopping mechanism which is a component of the positioning algorithm described above. It has been found that each magnet in a single-use probe may vary slightly from its specified strength. As described above, the positioning algorithm uses the Hall effect sensor 245 to continuously monitor the field strength of magnets 255a and 255b as the drive coupling 150 rotates and the algorithm determines the rotational position of the magnets and drive coupling based on the field strength, with the field strength rising and falling as a magnet rotates past the Hall sensor. Thus, it is important for the algorithm to have a library of fields strengths that accurately correspond to degrees of rotation away from a peak Hall signal when a magnet is adjacent the sensor 245. For this reason, an initial step of the positioning algorithm includes a “learning” step that allow the controller to learn the actual field strength of the magnets 255a and 255b which may vary from the specified strength. After a new single-use probe 110 (FIG. 1) is coupled to the handle 104, and after actuation of the motor drive 105, the positioning algorithm will rotate the drive coupling at least 180° and more often at least 360° while the Hall sensor 245 quantifies the field strength of the particular probe's magnets 255a and 255b. The positioning algorithm then stores the maximum and minimum Hall signals (corresponding to North and South poles) and calibrates the library of field strengths that correspond to various degrees of rotation away from a Hall min-max signal position when a magnet is adjacent the Hall sensor.

In general, a method of use relating to the learning algorithm comprises providing a handle with a motor drive, a controller, and a probe with a proximal hub configured for detachable coupling to the handle, wherein the motor drive is configured to couple to a rotating drive coupling in the hub and wherein the drive coupling carries first and second magnets with North and South poles positioned differently relative to said axis, and coupling the hub to the handle, activating the motor drive to thereby rotate the drive coupling and magnets at least 180°, using a handle sensor to sense the strength of each magnet, and using the sensed strength of the magnets for calibration in a positioning algorithm that is responsive to the sensor sensing the varying strength of the magnets in the rotating drive coupling to thereby increase accuracy in calculating the rotational position of the drive coupling 150.

Another aspect of the invention relates to an enhanced method of use using a probe working end with an electrode, such as the working end 112 of FIGS. 1 and 3B. As described above, a positioning algorithm is used to stop rotation of the electrode 155 in the default centerline position of FIG. 3B. An additional “slight oscillation” algorithm is used to activate the motor drive 105 contemporaneous with RF current to the electrode 155, particularly an RF cutting waveform for tissues ablation. The slight oscillation thus provides for a form of oscillating RF ablation. The slight oscillation algorithm rotates the electrode 155 in one direction to a predetermined degree of rotation, which the controller algorithms determine from the Hall position signals. Then, the algorithm reverses direction of the motor drive to rotate in the opposite direction until Hall position signals indicate that the predetermined degree of rotation was achieved in the opposite direction away from the electrode's default centerline position. The predetermined degree of angular motion can be any suitable rotation that is suitable for dimensions of the outer sleeve window, and in one variation is from 1° to 30° in each direction away from the centerline default position. More often, the predetermined degree of angular motion is from 5° to 15° in each direction away from the centerline default. The slight oscillation algorithm can use any suitable PID controlled motor shaft speed, and in one variation the motor shaft speed is from 50 RPM to 5,000 RPM, and more often from 100 RPM to 1,000 RPM. Stated another way, the frequency of oscillation can be from 20 Hz to 2,000 Hz and typically between 40 Hz and 400 Hz.

While the above description of the slight oscillation algorithm is provided with reference to electrode 155 on a rotating cutting member 145 of FIG. 3B, it should be appreciated that a reciprocating electrode 212 as shown in the working end 200C of FIG. 6 end could also be actuated with slight oscillation. In other words, the hook shape electrode 212 of FIG. 6 could be provided with a frequency of oscillation ranging from 20 Hz to 2,000 Hz and typically between 40 Hz and 400 Hz.

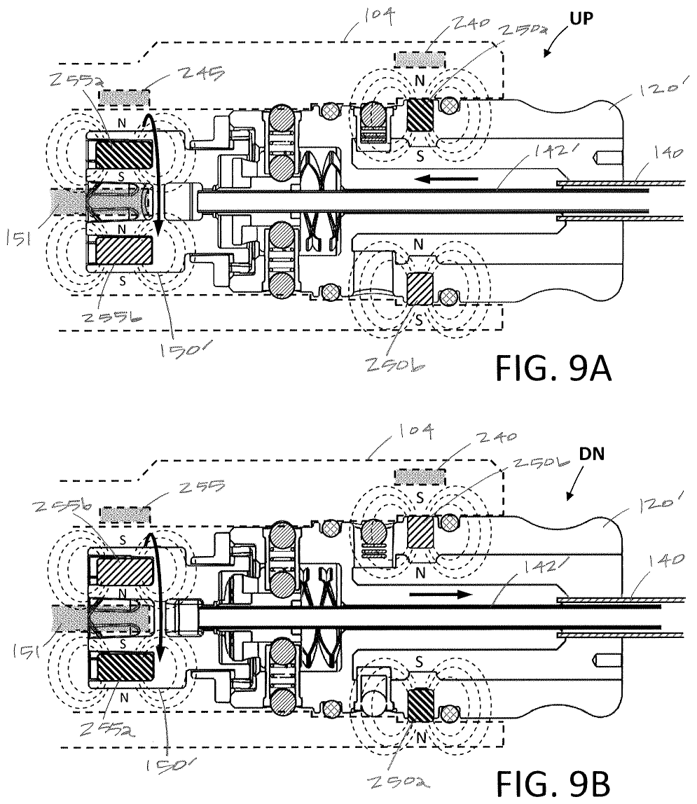

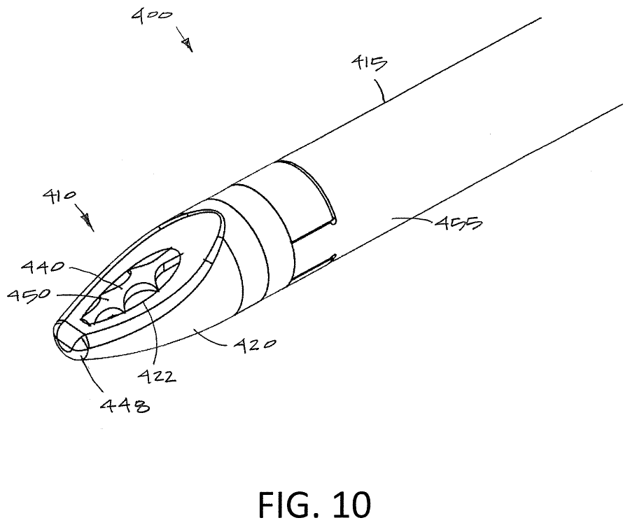

FIGS. 9A-9B are longitudinal sectional views of a probe hub 120′ that corresponds to the working end 200B of FIG. 5 which has a reciprocating electrode 210. In FIGS. 9A-9B, the handle 104 and Hall effect sensors 240 and 245 are of course the same as described above as there is no change in the handle 104 for different types of probes. The probe hub 120′ of FIGS. 9A-9B is very similar to the hub 120 of FIGS. 2A-2B with the first and second identification/orientation magnets 250a and 250b being the same. The third and fourth rotation al position magnets 255a and 255b also are the same and are carried by drive coupling 150′. The probe hub 120′ of FIGS. 9A-9B only differs in that the drive coupling 150 rotates with a cam mechanism operatively coupled to inner sleeve 142′ to convert rotational motion to linear motion to reciprocate the electrode 210 in working end 200B of FIG. 5. A similar hub for converting rotational motion to linear motion is provided for the working ends 200C and 200D of FIGS. 6 and 7, respectively, which each have a reciprocating component (212, 218) in its working end.

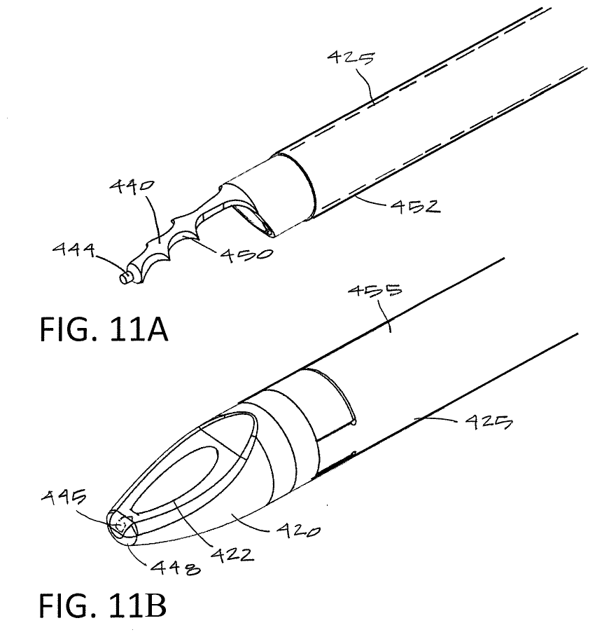

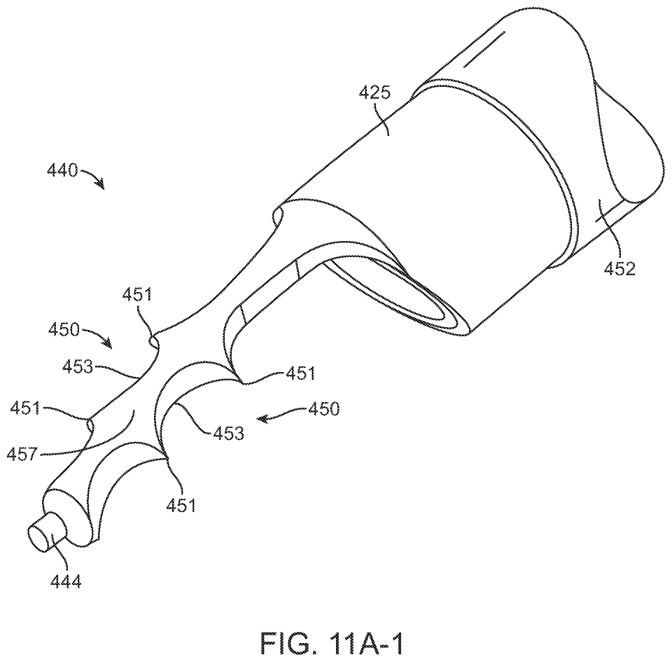

FIG. 10 is a perspective view of another variation of a probe 400 with a working end 410 wherein the probe 400 again may be detachably coupled to the handle of FIG. 1. In this variation, working end 410 has an outer sleeve 415 coupled to a distal ceramic member 420 with a cutting window 422 therein for receiving tissue. A motor-driven inner sleeve 425 (FIG. 11A) carries a distal electrode 440 that is adapted to rotate within the outer cutting window 422 in the ceramic member 420.

FIG. 11A shows the inner sleeve 425 and distal electrode 440 of FIG. 10 separated from the outer ceramic member 420. FIG. 11B illustrates the outer ceramic member 420 of FIG. 10 separated from the rotating inner electrode member 440. As can be understood from FIG. 11A, the electrode member 440 has a distal pin 444 that is insertable into a receiving bore 445 in the interior of a distal tip 448 of the ceramic member 420 (FIG. 11B). Thus, the electrode member 440 can rotate or rotationally oscillate at high speeds within the ceramic member about the distal pin 444 which defines a rotational “pivot” or bearing. Advantageously, the active electrode surface 450 of the electrode member 440 is limited to a side, preferably a single side, allowing the RF source to more easily ignite a plasma for tissue cutting about the electrode surface 450, which would not be the case if the electrode surface extended over all surface area of the cutting member. In general, it has been found that if the active electrode surface 450 has an area of less than 10 mm², usually less than 8 mm² area, and preferably less than 6 mm², the RF source can efficiently ignite plasma which will not be extinguished at high speed movement in saline as it rotates. Exemplary electrode surface area ranges include 1 mm² to 10 mm²; 1 mm² to 8 mm²; 1 mm² to 6 mm²; 2 mm² to 10 mm²; 2 mm² to 8 mm²; and 2 mm² to 6 mm².

In FIG. 11A, it can be seen that the inner sleeve 425 is covered with an insulator sleeve 452 so that it is entirely insulated from the outer sleeve 425 and the ceramic member 420. Thus, a portion of the exterior surface of the outer sleeve 425 can provide a return electrode 455.

In the exemplary embodiment of FIGS. 10 and 11A, as best seen in the enlarged view of FIG. 11A-1, each electrode surface 450 on the distal electrode 440 is serrated with at least one and preferably at least two sharpened tips 451 separated by intermediate notches 453. The electrode surface is chamfered relative to an axially aligned backbone 457 of the distal electrode 440. While the preferred embodiments of the distal electrode will be generally symmetrical about a longitudinal axis defined by the backbone 457, thus allowing for bidirectional rotation or rotational oscillation of the electrode past the cutting window 422 in the outer sleeve 415 (FIG. 10), in some instances, the serrated electrode surfaces 450 on each side may have different structures for different purposes or may be provided on only one side of the electrode if unidirectional rotation is sufficient.

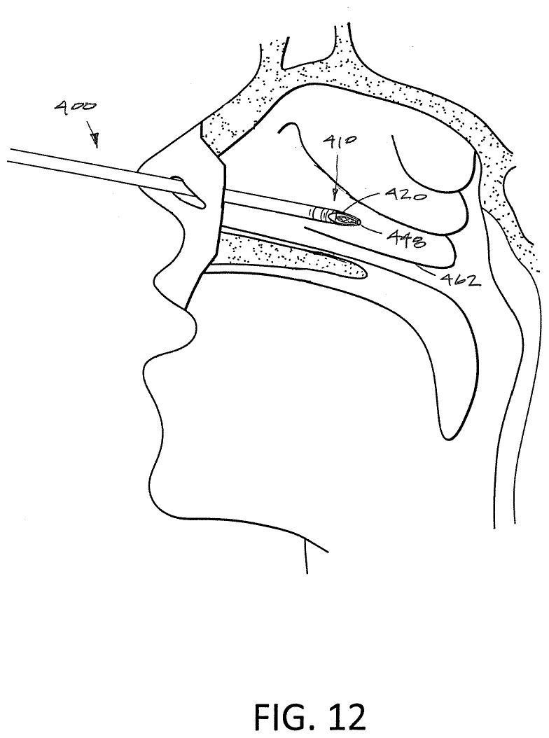

FIG. 12 is a schematic sectional view of a patient's nasal cavities 460 showing the working end 410 of the probe 400 of FIG. 10 being used to remove tissue in a turbinate 462. In use, the distal tip 448 of the ceramic member 420 may be sharp for penetrating the targeted tissue or the tip can be somewhat blunt in which case the working end may be activated to cut its way into the interior of the tissue, e.g. including an RF cutting tip of a type well known in the art (not shown). As can be understood in FIG. 12, fluid inflow may be desirable during the tissue resection process wherein such a fluid inflow can be provided in any known manner, e.g. by gravity from a fluid source or from a positive pressure source. Such fluid inflow can be provided through an introducer sleeve (not shown) or through an annular space or channel between the inner sleeve 425 and the outer sleeves 415 to irrigate the treatment site. In such an ENT procedure, the physician may view the treatment site with an endoscope (not shown) or in some cases can rely on direct observation.

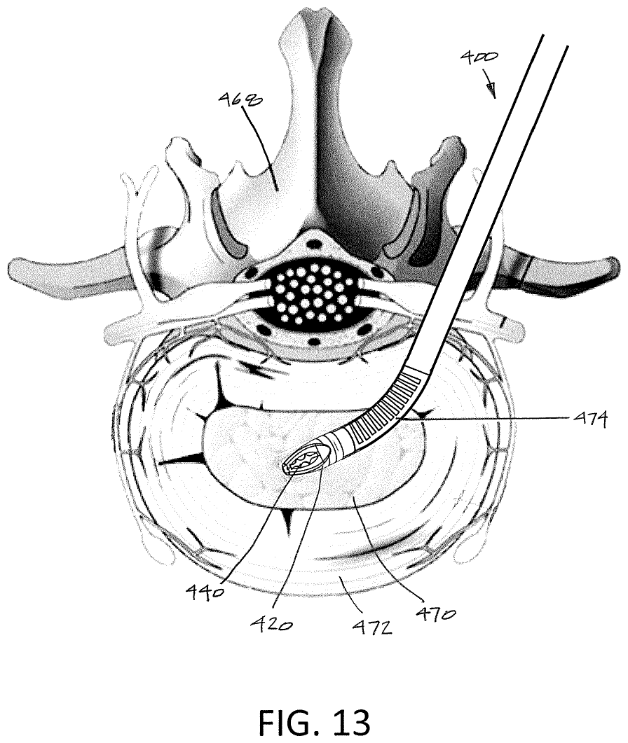

As shown in FIG. 13, a probe 400 may optionally have a curved, bendable, or steerable portion 474 at a distal end of the probe immediately proximal of the ceramic member 420 and be used in discectomy procedures, i.e., be used to remove nucleus tissue 470 from a spinal disc 472. In such a spinal procedure, access to the disc nucleus can be made through access methods known in the art and may use an endoscope.

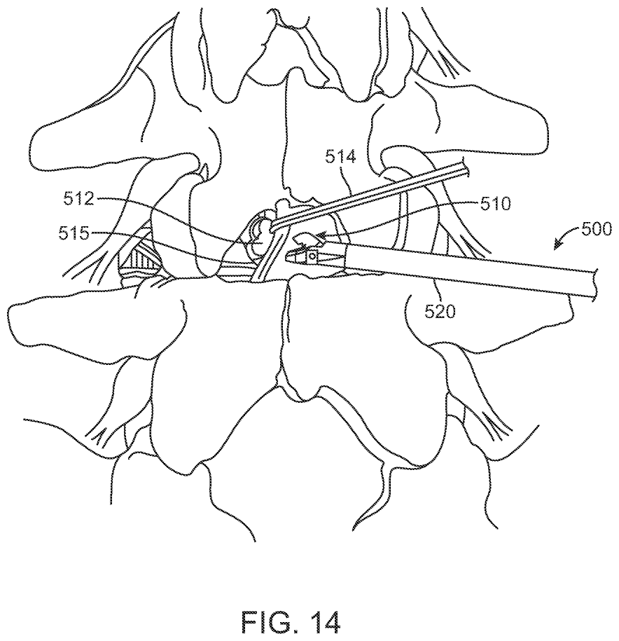

FIG. 14 is a schematic view of a patient's spine showing a probe 500 with a working end 510 similar to that of FIG. 7 being used in a discectomy procedure to remove tissue from a torn or herniated disc 512. In this example, a retracting device 514 is shown moving nerve tissue 515 away from the treatment site to provide access to the target disc 512. As described above, the working end 510 is motor driven to take individual small bites from the herniated disc tissue 512. In one variation, the working end 510 includes an electrosurgical jaw to assist in cutting tissue which also can use as a coagulation instrument. Again, the physician may view the treatment site through an open incision or an endoscope. Irrigation may be provided as needed either through the probe shaft 520 as described above or through an introducer sleeve or cannula.

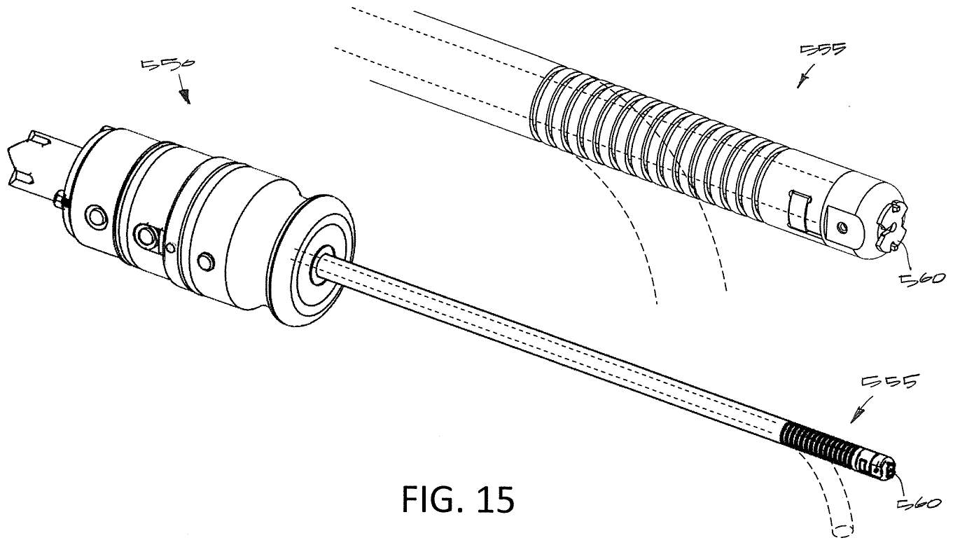

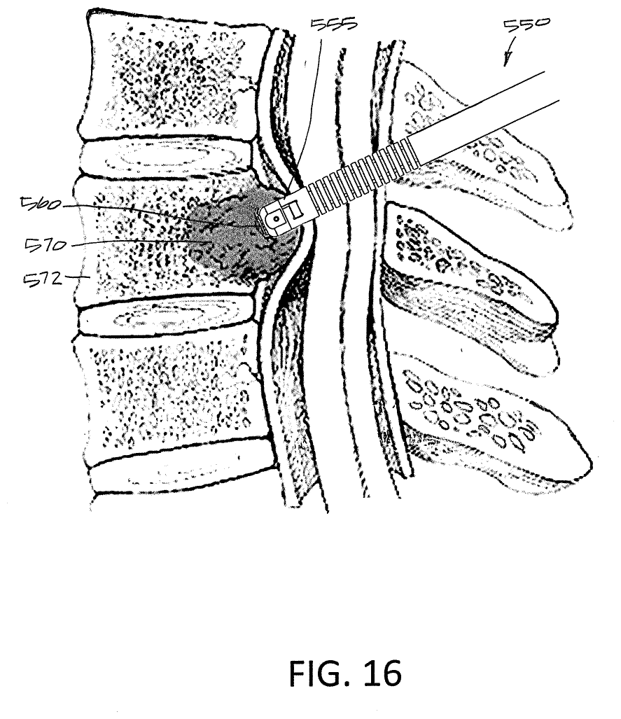

FIG. 15 illustrates another variation of a probe 550 with a working end 555 that carries an electrode arrangement 560 wherein the working end can be articulated with the motor drive. An articulating probe has been disclosed in connection with the device of FIG. 6 which is described in more detail in co-pending and commonly owned patent application Ser. No. 15/454,342 filed Mar. 9, 2017 (Atty. Docket 41879-715.201) titled ARTHROSCOPIC DEVICES AND METHODS. Again, the probe may be detachably coupled to the handle of FIG. 1. In one variation, a bipolar electrode arrangement is provided for ablating tumors such as spinal tumor 570 in vertebrae 572 as shown in FIG. 16.

Although particular embodiments of the present invention have been described above in detail, it will be understood that this description is merely for purposes of illustration and the above description of the invention is not exhaustive. Specific features of the invention are shown in some drawings and not in others, and this is for convenience only and any feature may be combined with another in accordance with the invention. A number of variations and alternatives will be apparent to one having ordinary skills in the art. Such alternatives and variations are intended to be included within the scope of the claims. Particular features that are presented in dependent claims can be combined and fall within the scope of the invention. The invention also encompasses embodiments as if dependent claims were alternatively written in a multiple dependent claim format with reference to other independent claims.

Other variations are within the spirit of the present invention. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A tissue treatment device comprising:
a sleeve assembly having an outer sleeve and an inner sleeve co-axially and rotatably received in an axial lumen of the outer sleeve;
a tapered ceramic member having a cutting window formed on a side surface thereof attached to a distal end of the outer member;
a distal electrode having at least one serrated electrode surface disposed along at least one axially aligned edge thereof so that said at least one serrated electrode surface passes across the cutting window in the tapered ceramic member as the inner sleeve rotated in the outer sleeve; and
a hub attached to a proximal end of the sleeve assembly, wherein said hub is configured to be detachably received in a motorized handle.

2. The tissue treatment device of claim 1, wherein the tapered ceramic member has a generally conical shape and the cutting window has an ovoid periphery.

3. The tissue treatment device of claim 2, wherein the distal electrode has an axial backbone with the at least one serrated electrode surface disposed along at least one axially side thereof

4. The tissue treatment device of claim 3, wherein the axial backbone is curved to conform to an inner curved surface of the tapered ceramic member as the electrode is rotated.

5. The tissue treatment device of claim 4, wherein the distal electrode includes two serrated electrode surfaces disposed symmetrically on each lateral side of the axial backbone.

6. The tissue treatment device of claim 1, wherein the at least one serrated electrode surface has an active area no greater than 10 mm².

7. The tissue treatment device of claim 6, wherein the active area is no less than 1 mm².

8. The tissue treatment device of claim 7, wherein the active area in any one of the following ranges: 1 mm² to 10 mm²; 1 mm² to 8 mm²; 1 mm² to 6 mm²; 2 mm² to 10 mm²; 2 mm² to 8 mm²; and 2 mm² to 6 mm².

9. A surgical system comprising;
the tissue treatment device of claim 1;
a handle including a motor attachable to the hub, said motor configured to rotatably drive the inner sleeve relative to the outer sleeve;
a radiofrequency (RF) current source configured to be coupled to the at least one distal electrode; and
a controller configured to be operatively coupled to the motor in the handle and to the RF source.

10. A method for performing a discectomy in a patient, said method comprising:
providing a tissue treatment device including:
a sleeve assembly having an outer sleeve and an inner sleeve co-axially and rotatably received in an axial lumen of the outer sleeve;
a tapered ceramic member having a cutting window formed on a side surface thereof attached to a distal end of the outer member;
a distal electrode having at least one serrated electrode surface disposed along at least one axially aligned edge thereof so that said at least one serrated electrode surface passes across the cutting window in the tapered ceramic member as the inner sleeve rotated in the outer sleeve; and
performing the following steps:
advancing the tapered ceramic member into a spinal disc of the patient;
rotating the inner sleeve relative to the outer sleeve to advance the at least one serrated electrode surface past the cutting window; and
applying radiofrequency current to the at least one serrated electrode surface to ablate tissue of the disc as the inner sleeve is being rotated.

11. A method as in claim 10, wherein the inner sleeve is rotated in one direction.

12. A method as in claim 10, wherein the inner sleeve is rotated in two direction.

13. A method as in claim 10, wherein the inner sleeve is rotationally oscillated.

14. The method of claim 10, wherein the tapered ceramic member has a generally conical shape and the cutting window has an ovoid periphery.

15. The method of claim 14, wherein the distal electrode has an axial backbone with the at least one serrated electrode surface disposed along at least one axially side thereof

16. The method of claim 15, wherein the axial backbone is curved to conform to an inner curved surface of the tapered ceramic member as the electrode is rotated.

17. The method of claim 16, wherein the distal electrode includes two serrated electrode surfaces disposed symmetrically on each lateral side of the axial backbone.

18. The method of claim 10, wherein the at least one serrated electrode surface has an active area no greater than 10 mm².

19. The method of claim 18, wherein the active area is no less than 1 mm².

20. The method of claim 19, wherein the active area in any one of the following ranges: 1 mm² to 10 mm²; 1 mm² to 8 mm²; 1 mm² to 6 mm²; 2 mm² to 10 mm²; 2 mm² to 8 mm²; and 2 mm² to 6 mm².
